(19) United States
(12) Patent Application Publication
(10) Pub. No.: US 2025/0290836 A1
(43) Pub. Date: Sep. 18, 2025

(54) SAMPLE PREPARATION DEVICE AND METHODS OF USE THEREOF

(71) Applicant: Akonni Biosystems, Inc., Frederick, MD (US)

(72) Inventors: Christopher G. COONEY, Frederick, MD (US); Arial BUENO, Frederick, MD (US); Ryan NORVILLE, Frederick, MD (US); Yvonne LINGER, Frederick, MD (US); Jon DAVIS, Frederick, MD (US)

(73) Assignee: Akonni Biosystems, Inc., Frederick, MD (US)

(21) Appl. No.: 19/082,369

(22) Filed: Mar. 18, 2025

Related U.S. Application Data
(60) Provisional application No. 63/566,456, filed on Mar. 18, 2024.
(60) Provisional application No. 63/661,756, filed on Jun. 19, 2024.

(51) Int. Cl. G01N 1/40; C12N 15/10; G01N 1/14

(52) U.S. Cl. CPC G01N 1/4077; C12N 15/1003; G01N 1/14; G01N 2001/1454; G01N 2001/4088

(57) ABSTRACT

A syringe-like sample preparation device is disclosed. The syringe-like sample preparation device comprises a syringe body having a proximate end, a distal end and a syringe body cavity between the proximate end and the distal end; a plunger located at the proximate end of the syringe body and movable along the syringe body cavity; and an overflow tube at the distal end of the syringe body and extending into the body cavity of the syringe body; and a sample purification tip at the distal end of the syringe body, wherein the sample purification tip comprises a sample binding matrix.

This application claims priority from U.S. Provisional App. No. 63/566,456, filed Mar. 18, 2024 and U.S. Provisional App. No. 63/661,756, filed Jun. 19, 2024, which are incorporated herein by reference.

### FIELD

The present application generally relates to sample preparation devices in the biochemical art and, in particular, sample preparation devices using a porous binding matrix for sample filtration, separation, purification, and normalization.

### BACKGROUND

Many sample preparation devices have been developed in the biochemical art to purify/isolate the analyte of interest from a sample. However, there still exists a continuous need for sample preparation devices that can purify/isolate the analyte of interest from a sample with high efficiency at low cost.

### SUMMARY

One aspect of the application relates to a pipette tip-shaped sample preparation device that comprises (1) a pipette tip-shaped housing defining a passage-way between a first opening and a second opening, wherein the housing is configured in the shape of a pipette tip, and (2) a sample binding matrix occupying a section of the passage-way, wherein the sample binding matrix comprises composite frit comprising glass and a polymeric material.

Another aspect of the application relates to a syringe-shaped sample preparation device that comprises (1) a syringe body having a proximate end, a distal end and a syringe body cavity between the proximate end and the distal end, (2) a plunger located at the proximate end of the syringe body and movable along the syringe body cavity, (3) an overflow tube at the distal end of the syringe body and extending into the body cavity of the syringe body, and (4) a sample purification tip at the distal end of the syringe body, wherein the sample purification tip comprises a housing that defines a sample passage-way between a first opening and a second opening, wherein the second opening is located outside of the body cavity of the syringe body, and a sample binding matrix located within the sample passage way of the housing, wherein the sample passage-way of the housing is in fluid communication with the syringe body cavity through the overflow tube.

Another aspect of the application relates to a method for isolating an analyte from a biological sample with the pipette tip-shaped sample preparation device of the present application. The method comprises the steps of lysing the biological sample in a lysis solution to form a liquid lysate, passing the liquid lysate through the sample binding matrix of the pipette tip-shaped sample preparation device, wherein the analyte in the liquid lysate binds specifically to the sample binding matrix, washing the sample binding matrix with a washing solution, and releasing bound analyte from the sample binding matrix with an elution solution.

Another aspect of the application relates to a method for isolating an analyte from a biological sample with the syringe-shaped sample preparation device of the present application. The method comprises the steps of lysing the biological sample in a lysis solution to form a liquid lysate, passing the liquid lysate through the sample binding matrix of the syringe-shaped sample preparation device, wherein the analyte in the liquid lysate binds specifically to the sample binding matrix; washing the sample binding matrix with a washing solution; and releasing bound analyte from the sample binding matrix with an elution solution.

Another aspect of the application relates to a kit for isolating an analyte from a sample. The kit comprises a pipette tip-shaped sample preparation device of the present application, one or more syringes pre-filled with reagents needed for isolating the analyte from the sample; and instructions for how to use pipette tip-shaped sample preparation device with the pre-filled syringes.

Another aspect of the application relates to a kit for isolating an analyte from a sample. The kit comprises a syringe-shaped sample preparation device of the present application, a reagent pack; and instructions for how to use the syringe-shaped sample preparation device with the reagent pack.

### BRIEF DESCRIPTION OF DRAWINGS

The figures herein are illustrative of non-limiting embodiments of the invention. The detailed description will refer to the following drawings, wherein like numerals refer to like elements, and wherein:

FIG. 1 shows an embodiment of a pipette-tip type sample preparation device.

FIG. 2 shows an embodiment of a syringe-type sample preparation device.

FIG. 3 shows an embodiment of a reagent pack.

FIG. 4 shows a dual-acting syringe-type sample preparation device for automated sample preparation.

FIG. 5 shows a dual-acting syringe-type sample preparation system. The system allows for separate controls for course (large) volume and fine (small) volume aspirations.

FIG. 6 is a demonstration of the binding matrix for normalization from PCR amplified DNA. The plateau illustrates that the binding matrix is saturated to allow normalization of the DNA concentrations of the PCR-amplified libraries above this concentration.

FIG. 7 is a composite of bioanalyzer electropherograms. Top Panel: Prior to PCR amplification of M13 DNA; strong peaks from the primers can be seen. Middle Panel: After PCR, before Normalization/Cleanup; primer-dimer peak is present because of the absence of a cleanup step. Bottom Panel: After PCR, and after Normalization/Cleanup; no peaks other than the 140 base pair (bp) amplicon product is present. These results demonstrate the potential of a single binding matrix in a pipette tip to simultaneously normalize (reduction in signal) and perform PCR cleanup (shows a single clean peak).

### DETAILED DESCRIPTION

The aspects of the application are described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

### I. Definitions

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it is understood that the particular value forms another embodiment. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

The term “binding matrix,” as used herein, refers to a porous material that is capable of binding to an analyte of interest.

The term “composite” as used herein, refers to a mixture of two or more different materials. For example, a composite may contain glass beads and a polymeric material, such as a copolyester.

The term “frit” “monolith”, “monolithic frit”, or “monolithic material,” as used in this application, refers to a porous, three-dimensional material having a continuous interconnected pore structure in a single piece. A frit or monolith is prepared, for example, by casting, heating, sintering, frittage or polymerizing precursors into a mold of a desired shape. Sintering or frittage is the process of compacting and forming a solid mass of material by pressure or heat without melting it to the point of liquefaction. Sintering happens as part of a manufacturing process used with metals, ceramics, glasses, plastics, polymers and mixtures thereof. The terms “monolith” and “frit” are used interchangeably in this application.

The term “monolith adsorbent” or “monolithic adsorbent material” is meant to be distinguished from a collection of individual adsorbent particles packed into a bed formation or embedded into a porous matrix, in which the end product comprises individual adsorbent particles. The term “monolith adsorbent” or “monolithic adsorbent material” is also meant to be distinguished from a collection of adsorbent fibers or fibers coated with an adsorbent, such as binding matrix papers or binding matrix papers coated with an adsorbent.

The term “porosity,” refers to a measure of the void spaces in a material and is a fraction of the volume of voids over the total volume, between 0 and 1, or as a percentage between 0% and 100%. Strictly speaking, some tests measure the “accessible void”, the total amount of void space accessible from the surface.

The term “pore size” refers to the mean of the diameters of a population of pores in which the diameter is the distance between the furthest two points on a cross section of a pore.

The term “specifically bind to” or “specific binding,” as used in the embodiments described hereinafter, refers to the binding of the adsorbent to an analyte (e.g., nucleic acids) with a specificity that is sufficient to differentiate the analyte from other components or contaminants of a sample. In one embodiment, the dissociation constant of the adsorbent/ligand complex is less than about 1×10⁻⁶ M. A person of ordinary skill in the art understands that stringency of the binding and elution of the analyte to the adsorbent can be controlled by binding and elution buffer formulations. For example, elution stringencies for nucleic acids can be controlled by salt concentrations using KCl or NaCl. Nucleic acids, with their higher negative charge, are more resistant to elution than proteins. Temperature, pH, and mild detergent are other treatments that could be used for selective binding and elution. Thermal consistency of the binding and elution may be maintained with a heat block or a water bath. The manipulation of the binding buffer is preferable since the impact of the modified elution buffer on the downstream analyzer would need to be evaluated.

The term “nucleic acid,” as used in the embodiments described hereinafter, refers to individual nucleic acids and polymeric chains of nucleic acids, including DNA and RNA, whether naturally occurring or artificially synthesized (including analogs thereof), or modifications thereof, especially those modifications known to occur in nature, having any length. Examples of nucleic acid lengths that are in accord with the present invention include, without limitation, lengths suitable for PCR products (e.g., about 50 to 700 base pairs (bp)) and human genomic DNA (e.g., on an order from about kilobase pairs (kb) to gigabase pairs (Gb)).

Thus, it will be appreciated that the term “nucleic acid” encompasses single nucleic acids as well as stretches of nucleotides, nucleosides, natural or artificial, and combinations thereof, in small fragments, e.g., expressed sequence tags or genetic fragments, as well as larger chains as exemplified by genomic material including individual genes and even whole chromosomes. The terms “prevent”, “preventing” or “prevention,” as used herein, refer to a method of barring a subject from acquiring a disorder and/or its attendant symptoms. In certain embodiments, the terms “prevent,” “preventing” or “prevention” refer to a method of reducing the risk of acquiring a disorder and/or its attendant symptoms.

### II. Sample Preparation Device

One aspect of the present application relates to a sample preparation device. The sample preparation device of the present application comprises a housing and a sample binding matrix. The housing defines a sample passage-way between a first opening and a second opening. The shape and size of the housing are not particularly limited. The preferred housing configuration is substantially cylindrical so that the flow velocity vectors during operation do not intersect, resulting in laminar flow thereby minimizing or avoiding dilutional washing that might occur with non-cylindrical configurations. The sample binding matrix is located inside the housing and binds specifically to an analyte of interest.

### A. Sample Preparation Device With Tip/Column Configuration

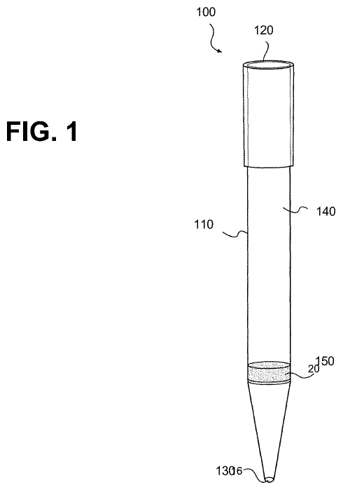

FIG. 1 shows an embodiment of the sample preparation device of the present application. In this embodiment, the sample preparation device 100 has a configuration of a pipette tip. The housing 110 has a pipette tip geometry with the first opening 120 having a diameter that is greater than the diameter of the second opening 130, and the first opening 120 is dimensioned to fit onto the tip of a pipettor or a syringe. The first opening 120 is in fluid communication with the second opening 130 through the passage-way 140. The sample binding matrix 150 is placed in close proximity to the second opening 130 so that target molecules in a sample may bind to the binding matrix immediately after being transported into the housing 110 through the second opening 130. In one embodiment, the sample binding matrix 150 is contiguous with the second opening 130. In another embodiment, the sample binding matrix 150 is separated from the second opening 130 by a distance of 1-20 mm. In another embodiment, the housing 110 has a column geometry with similar diameters throughout the housing 110. In some embodiments, the sample binding matrix 150 is a sintered frit made of composite materials.

In some embodiments, the housing contains an aerosol binding matrix in the proximity of the first opening to prevent contamination from a pumping device. In some embodiments, the housing further contains a plurality of mechanical lysing beads, such as glass beads, in the space between the sample binding matrix and the aerosol binding matrix. The mechanical lysing beads are used to disrupt the cells and release the nucleic acid by vortexing the entire sample preparation device. In this embodiment, the second opening may be covered with a cap during vortexing to prevent the liquid from escaping from the second opening.

### B. Sample Preparation Device With Syringe Configuration

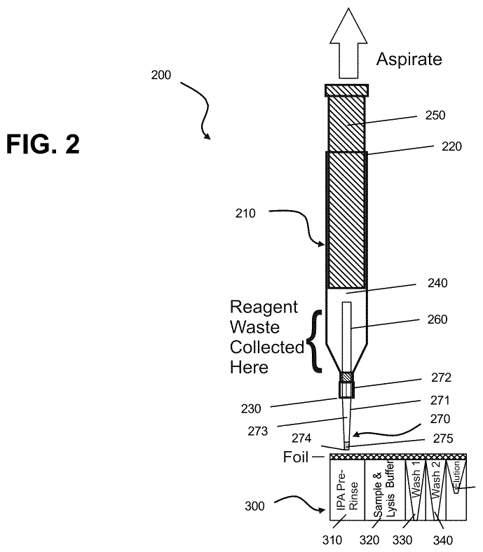

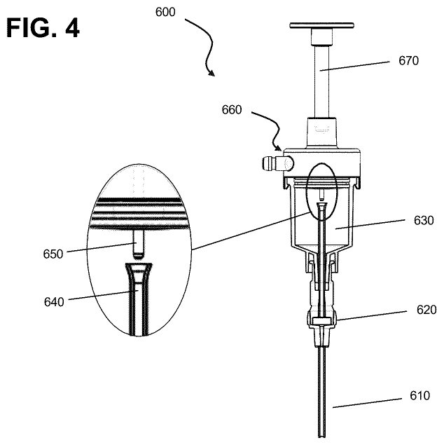

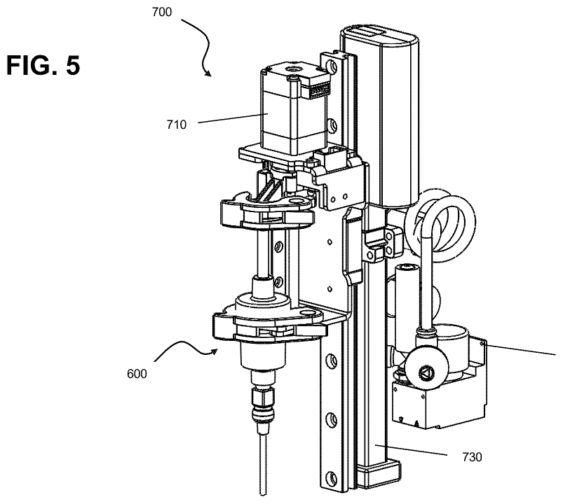

FIG. 2 shows another embodiment of the sample preparation device. In this embodiment, the sample preparation device 200 has a syringe-like shape and comprises (1) a syringe body 210 having a proximate end 220, a distal end 230 and a syringe body cavity 240 between the proximate end 220 and the distal end 230, (2) a plunger 250 located at the proximate end 220 of the syringe body 210 and movable along the syringe body cavity 240, and (3) an overflow tube 260 at the distal end 230 and extending into the body cavity 240 of the syringe body 210, and (4) a removable or non-removable sample purification tip 270 at the distal end 230 of the syringe body 210. The sample purification tip 270 may have the same configuration as the sample preparation device 100 shown in FIG. 1. In some embodiments, The sample purification tip 270 has a column-like configuration (e.g., FIG. 4 and FIG. 5)

In some embodiments, the sample purification tip 270 comprises (A) a housing 271 that defines a sample passage way 273 between a first opening 272 and a second opening 274 and (B) a sample binding matrix 275 located within the sample passage way 273 of the housing 271. The first opening 272 is connected to the distal end 230 of the syringe body 210. The second opening 274 is distal to the distal end 230 of the syringe body 210 and is located outside of the syringe body cavity 240. In some embodiments, the sample binding matrix 275 of the sample purification tip 270 is located near or at the second opening 274. Also shown in FIG. 2 is an embodiment of a Blister reagent pack 300 that contains a chamber 310 for isopropanol (IPA) pre-rinse solution, a chamber 320 for sample and lysis solution, chambers 330 and 340 for wash solutions, and a chamber 350 for the elution buffer.

In some embodiments, the second opening of the housing extends about 10-80 mm, 10-60 mm, 10-40 mm, 20-80 mm, 20-60 mm or 20-40 mm from the distal end into the syringe body, so as to form a waste collection space 280 within the syringe body cavity 240.

The syringe-type sample preparation device can be a one-barrel syringe, two-barrel syringe, or multiple barrel syringes allowing for multiple aspirations into different barrels or to allow dispensing at different intervals. The syringes can include pre-loaded liquids such as bind buffers, wash buffers, and elution buffers. The syringes can be separated or integrated. The plungers can be connected or separated. Aspiration can also be performed with a pipette, which includes a manual pipette, an electronic pipette, a serological pipette, or automated liquid handling platforms. The syringes can be combined with automated mechanisms for aspirating and dispensing into reagent trays or reagent packages/discs (see, e.g., Example 4). The steps can include sample preparation steps and/or library preparation steps. This can be automated by implementing either or both of two design approaches: a single dual-acting syringe that interfaces with a serological-style (diaphragm) pump for course volume control and a precision piston (stepper motor) for fine volume control (see e.g., Example 4). The use of a diaphragm pump allows for high flow rates without an end limit (unlike conventional pipettes) which is beneficial for air drying; it has high linearity over a wide range of flow rates, which is advantageous for the range of volumes required for this application; and it does not require a plunger allowing for a small form factor. During sample extraction, the small piston (shown in the inset of FIG. 4) does not enter the overflow tube, thus the diaphragm pump controls the flow rate across the binding frit. When extraction is complete, a precision stepper motor advances the piston into the overflow tube and allows for fine volume control for sample analysis. When the protocol is complete the disposable dual-acting syringe retains the waste. In some embodiments, the reagent pack is designed to interface with a thermoelectric heater-cooler.

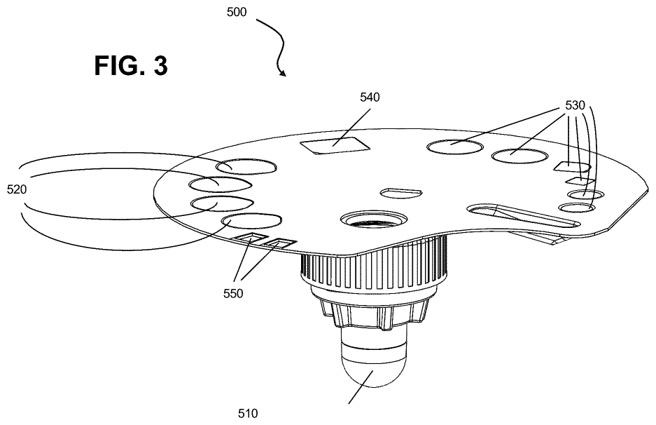

FIG. 3 shows an embodiment of a reagent pack of the present application. In this embodiment, the reagent pack 500 is a Blister-type reagent pack that comprises a lysis & sample tube 510 that contains a lysis solution for lysing tissues/cells/microorganisms in a biological sample, wells 520 that contain wash solutions, wells 530 that contain elution solution and reagents for DNA library preparation, a mixing chamber 540 that interfaces with heater/cooler for sample preparation/analyte amplification (e.g., PCR reaction), and a coding cutout or marking 550 for identification of the type of the reagent pack.

The syringe-type device of the present application allows for a field-portable method of nucleic acid extraction without needing a separate waste container. The user adds the sample to a sample well in a reagent pack (e.g., FIG. 3), and then mixes by hand the sample with the reagent in the sample well. The user then progresses through each of the reagent wells in the reagent pack, aspirating all of the reagents into the syringe. The sample and bind buffer flow up the overflow tube and into the bottom of the syringe when held upright. If necessary, the user can depress the syringe again (without dispensing liquid in the syringe) with the syringe held upright. After aspirating the solution from the last well, the user aspirates the elution buffer just past the binding matrix, and then dispenses it into the proper receptacle or test strip. The sample is purified and ready for downstream molecular analysis.

### Size and Material of the Housing

In some embodiments, the housing 110 or 271 has a volume of about 1 μl to about 10 ml. In another embodiment, the housing 110 or 271 has a volume of about 10 μl to about 10 ml. In another embodiment, the housing 110 or 271 has a volume of about 100 μl to about 10 ml. In another embodiment, the housing 110 or 271 has a volume of about 100 μl to about 5 ml. In another embodiment, the housing 110 or 271 has a volume of about 100 μl to about 2 ml. In another embodiment, the housing 110 or 271 has a volume of about 200 μl to about 2 ml. In another embodiment, the housing 110 or 271 has a volume of about 100 μl to about 1 ml. In another embodiment, the housing 110 or 271 has a volume of about 100 μl to about 1.2 ml.

In some embodiments, the syringe body 210 has a volume of about 1 ml to about 20 ml. In some embodiments, the syringe body 210 has a volume of about 1 ml to about 10 ml. In some embodiments, the syringe body 210 has a volume of about 1 ml to about 5 ml. In some embodiments, the syringe body 210 has a volume of about 2 ml to about 20 ml. In some embodiments, the syringe body 210 has a volume of about 2 ml to about 10 ml. In some embodiments, the syringe body 210 has a volume of about 2 ml to about 5 ml. In some embodiments, the syringe body 210 has a volume of about 2 ml to about 3 ml. In some embodiments, the syringe body 210 has a volume of about 1 ml to about 2 ml. In some embodiments, the syringe body 210 has a volume of about 0 ml to about 1 ml.

Suitable materials for the housing (e.g., 110 or 271) or the syringe body (e.g., 210), are not particularly limited. Examples include, but are not limited to, plastics (such as polypropylene, polyethylene, cyclic olefin polymer, and cyclic olefin copolymer), glass and stainless steel.

### Size, Location and Material of the Sample Binding Matrix

The sample binding matrix (e.g., 150 and 275) can be made of any porous monolithic material that binds specifically to an analyte of interest, such as nucleic acids. The porosity of the porous monolithic material is application dependent. In general, the porous monolithic material should have a porosity that allows for a desired sample flow rate for a particular application.

In some embodiments, the sample binding matrix is made of a finely porous glass frit through which a liquid sample may pass. Porous glass frits, which are sintered glass that begins with crushing beads in a hot press to form a single monolithic structure, are excellent substrates for purifying nucleic acids. The uniform structure of the frit provides predictable liquid flow inside the frit and allows the eluent to have similar fluid dynamics as the sample flows. The predictable liquid flow also leads to a higher recovery during the elution process.

In some embodiments, the binding matrix is a glass frit or composite frit with pore sizes between about 2 microns and about 220 microns. In one embodiment, the glass frit or composite frit has a pore size between about 2 microns and about 100 microns. In another embodiment, the glass frit or composite frit has a pore size between about 40 microns and about 75 microns. In another embodiment, the glass frit or composite frit has a pore size between about 150 microns and about 200 microns. In yet another embodiment, the glass frit or composite frit has a pore size between about 2 microns and about 20 microns. In yet another embodiment, the glass frit or composite frit has a pore size between about 20 microns and about 50 microns.

In some embodiments, the glass frit or composite frit has a thickness between about 1 mm and about 20 mm. In another embodiment, the glass frit or composite frit has a thickness between about 2 mm and about 6 mm. In yet another embodiment, the glass frit or composite frit as a thickness between about 3 mm and about 5 mm. In yet another embodiment, the glass frit or composite frit has a thickness of about 4 mm.

In some embodiments, the binding matrix is designed to intentionally saturate to normalize the DNA concentration to a specific amount so that all DNA concentrations across multiple samples are the same, which is common practice for library preparation in sequencing.

In some embodiments, the glass frit or composite frit is chemically treated to functionalize its surface.

For example, the glass frits may be derivatized with aminosilanes or treated with the ChargeSwitch® technology (Invitrogen, Carlsbad, CA) to create positive charges for better adsorption of the negatively charged nucleic acids.

In some embodiments, while the glass frit or composite frit is a good adsorbent for nucleic acids, a skilled artisan would recognize that the glass frit or composite frit may also be used to absorb other types of molecules. For example, the glass frit or composite frit may be coated with antibodies to extract other ligands of interest from the sample. In one embodiment, the glass frit is derivatized in polymethylmethacrylate (PMMA) and cyclic-olefin-copolymer (COC) with antibodies as capture moieties for microbes and toxin. The term “antibody”, as used herein, is used in the broadest possible sense and may include but is not limited to an antibody, a recombinant antibody, a genetically engineered antibody, a chimeric antibody, a monospecific antibody, a bispecific antibody, a multispecific antibody, a chimeric antibody, a heteroantibody, a monoclonal antibody, a polyclonal antibody, a camelized antibody, a deimmunized antibody, and an anti-idiotypic antibody. The term “antibody” may also include but is not limited to an antibody fragment such as at least a portion of an intact antibody, for instance, the antigen binding variable region. Examples of antibody fragments include Fv, Fab, Fab′, F(ab′), F(ab′)2, Fv fragment, diabody, linear antibody, single-chain antibody molecule, multispecific antibody, and/or other antigen binding sequences of an antibody. In another embodiment, the binding matrix is coated with lectins, which bind to carbohydrates found in bacteria coats and can be used to capture bacteria in a sample.

In some embodiments, the sample binding matrix is made of a porous glass monolith, a porous glass-ceramic, porous monolithic polymers or a porous composite frit. Porous glass monoliths may be produced using the sol-gel methods described in U.S. Pat. Nos. 4,810,674 and 4,765,818, which are hereby incorporated by reference. Porous glass-ceramic may be produced by controlled crystallization of a porous glass monolith. In some embodiments, the sample binding matrix is made of a frit of composite materials.

In some embodiments, porous monolithic polymers are a new category of materials developed during the last decade. In contrast to polymers composed of very small beads, a monolith is a single, continuous piece of a polymer prepared using a simple molding process.

In some embodiments, the housing serves as the mold for the porous monolithic polymers. Briefly, a section of the passage-way of the housing is filled with a liquid mixture of monomers and porogens. Next, a mask that is opaque to ultraviolet light is placed over the filled section. The mask has a small slit that exposes a small portion of the filled section. Finally, the monomers/porogens mixture in the filled section is irradiated with ultraviolet light through the tiny opening on the mask. The UV irradiation triggers a polymerization process that produces a solid but porous monolithic material in the filled section of the passage way.

In some embodiments, the sample binding matrix is made of a hydrophilic matrix with impregnated chemicals that lyses cell membranes, denaturing proteins, and traps nucleic acids.

In some embodiments, the hydrophilic matrix is FTA paper® (Whatman, Florham Park, NJ). Biological samples are applied to the FTA® paper and cells contained in the sample are lysed on the paper. The paper is washed to remove any non-DNA material (the DNA remains entangled within the paper). The DNA is then eluted for subsequent analysis.

The sample binding matrix is shaped to fit tightly into the passage-way to prevent the sample from channeling or bypassing the sample binding matrix during operation. In one embodiment, the binding matrix is fitted into the passage-way through mechanical means such as crimping, press fitting, and heat shrinking the housing or a portion thereof. In another embodiment, the binding matrix is attached to the interior of the passage-way through an adhesive. In yet another embodiment, the side of the binding matrix is tapered to have a frustoconical shape to the contour of the passage-way.

In some embodiments, the housing has the shape of a frustoconical pipette tip with the first opening dimensioned to fit on the end of a liquid delivery system, such as a manual pipettor or an electronic pipetting device. Samples are taken up though the second opening, passed through the sample binding matrix and then retained in the section of the housing that is above the sample binding matrix. In one embodiment, the liquid delivery system is an electronic pipetting device, such as an electronic pipettor or a robotic pipetting station.

In some embodiments, the sample binding matrix includes at least two sections, a first section that binds specifically to nucleic acids and a second section that specifically binds to another analyte of interest, such as proteins. In another embodiment, the housing contains a pre-binding matrix placed between the second opening and the sample binding matrix. The pre-binding matrix has a pore size that is larger than the pore size of the sample binding matrix and does not bind specifically to nucleic acids.

In some embodiments, the sample binding matrix is a binding matrix made from glass-reinforced, glass-filled or woven glass fiber, or from composites of glass and polymers, such as: polyethylene terephthalate, copolyesters, low-density polyethylene, high-density polyethylene, polyvinyl chloride, polypropylene, polystyrene, acrylonitrile-butadiene-styrene, acrylic or polymethyl methacrylate, polycarbonate, polyurethane, nylon, polyethylene terephthalate glycol, polyetheretherketone, polytetrafluoroethylene, polyamide, polylactic acid, polyoxymethylene, polyether block amide, ethylene vinyl acetate, polyimide, polyphenylene sulfide, polysulfone, polyetherimide, fluorinated ethylene propylene, polyvinylidene fluoride, styrene-acrylonitrile, polybutylene terephthalate, polymethylpentene, polyether sulfone, polyphenylene oxide, epoxy, vinylester, polyester thermosetting plastic, phenol formaldehyde resins, acetal homopolymers and copolymers, polyethylene oxide, liquid crystal polymers, polyethylenimine, ultra high molecular weight polyethylene, cyclic olefin polymer, and cyclic olefin copolymers. In some embodiments, the sample binding matrix is a composite frit. In some embodiments, the composite frit is prepared by sintering a composite of two or more polymer materials, or a composite of glass beads and one or more polymer materials.

In some embodiments composites are used to reduce the silica surface area to reduce the binding capacity for applications such as normalization (e.g. for library preparation for sequencing). In some embodiments, the amount of silica (e.g., glass) in the composite is in the range of 1-5 wt %, 1-10 wt %, 1-15 wt %, 1-25 wt %, 1-50 wt %, 1-75 wt %, 5-10 wt %, 5-15 wt %, 5-25 wt %, 5-50 wt %, 5-75 wt %, 10-15 wt %, 10-15 wt %, 10-25 wt %, 10-50 wt %, 10-75 wt %, 15-25 wt %, 15-50 wt %, 15-75 wt %, 25-50 wt %, 25-75 wt %, or 50-75 wt %. In some embodiments, the amount of polymer materials in the composite is in the range of 25-99 wt %, 25-95 wt %, 25-90 wt %, 25-85 wt %, 25-75% wt, 25-50 wt %, 50-99 wt %, 50-95 wt %, 50-90 wt %, 50-85 wt %, 50-75% wt, 75-99 wt %, 75-95 wt %, 75-90 wt %, 75-85 wt %, 85-99 wt %, 85-95 wt %, 85-90 wt %, 90-99 wt %, 90-95 wt %, or 95-99 wt %.

The use of glass-reinforced, a glass-filled, or woven glass fiber binding matrices or composites of glass and polymeric materials (composite glass binding matrices) can serve to provide the necessary binding surface sites for nucleic acid adsorption. In some embodiments, the pore sizes of the sample binding matrices are in the range of 1 micron to 100 microns, preferably in the range of 10 microns to 40 microns, 10 microns to 16 microns, 16 microns to 40 microns, or 40 microns to 50 microns. The thickness of the sample binding matrices are in the range of 0.5 mm to 10.0 mm, 1 mm to 8 mm, 2 mm to 6.0 mm, or 3 mm to 5 mm. In some embodiments, the sample binding matrices are constructed to bind specifically to nucleic acids, or nucleic acids of a desired size range under suitable ionic and pH conditions.

In some embodiments, the sample binding matrix consists of a single layer. In some embodiments, the sample binding matrix consists of a single monolith layer with two or more porous regions with different pore sizes. In some embodiments, the sample binding matrix consists of a single monolith layer with a first porous region and a second porous region, with pore sizes in the range of 2-20 microns, 2-15 microns, 2-10 microns, 5-20 microns, 5-15 microns, 5-10 microns, 7-20 microns, 7-15 microns, and 7-10 microns in the first regions and pore sizes in the range of 25-50 microns, 25-40 microns, 25-35 microns, 25-30 microns, 30-50 microns, 30-40 microns, 30-35 microns, 35-50 microns, 35-40 microns, or 40-50 microns in the second region. In some embodiments, the sample binding matrix consists of a single monolith layer with a first porous region and a second porous region, with pore sizes in the range of 1-10 microns in the first region and about 10-50 microns in the second region.

In some embodiments, the sample binding matrix is made from a blend of polymeric materials (such as polycarbonate and acrylonitrile-butadiene-styrene) and silica materials (such as glass beads or glass fibers). The polymer/silica blend is then heated or sintered to generate a single monolith binding matrix with two regions of pore sizes, as described above.

In some embodiments, the sample binding matrix is made by fusing two or more sintered glass or plastic monolithic frits of different pore size together. In some embodiments, the sample binding matrix consists of a first sintered glass monolithic frit (or composite) and a second sintered glass monolithic frit (or composite) that are fused together by heating or sintering. In some embodiments, the first sintered glass monolithic frit has pore sizes in the range of 2-20 microns, 2-15 microns, 2-10 microns, 5-20 microns, 5-15 microns, 5-10 microns, 7-20 microns, 7-15 microns, and 7-10 microns, and the second sintered glass monolithic frit has pore sizes in the range of 25-50 microns, 25-40 microns, 25-35 microns, 25-30 microns, 30-50 microns, 30-40 microns, 30-35 microns, 35-50 microns, 35-40 microns, or 40-50 microns. In some embodiments, the first sintered glass monolithic frit has pore sizes in the range of 1-10 microns and the second sintered glass monolithic frit has pore sizes about 10-50 microns.

Similarly, in some embodiments, the sample binding matrix has a conical shape that fits into the interior space of a pipette tip, wherein the second region has an average diameter that is smaller than the average diameter of the first region. In some embodiments, the sample binding matrix has a diameter in the range of 2-10 mm, 2-8 mm, 2-6 mm, 2-5 mm, 2-4 mm, 2-3 mm, 3-10 mm, 3-8 mm, 3-6 mm, 3-5 mm, 3-4 mm, 4-10 mm, 4-8 mm, 4-6 mm, 4-5 mm, 5-10 mm, 5-8 mm, 5-6 mm, 6-10 mm, 6-8 mm or 8-10 mm.

In some embodiments, the sample binding matrix has a thickness in the range of 0.5-10 mm. In some embodiments, the sample binding matrix has a thickness of 0.5-8.0 mm, 0.5-6.0 mm, 0.5-5.0 mm, 0.5-4.0 mm, 0.5-3.0 mm, 0.5-2.0 mm, 1.0-8.0 mm, 1.0-6.0 mm, 1.0-5.0 mm, 1.0-4.0 mm, 1.0-3.0 mm, 1.0-2.0 mm, 2.0-8.0 mm, 2.0-6.0 mm, 2.0-5.0 mm, 2.0-4.0 mm, 2.0-3.0 mm, 3.0-8.0 mm, 3.0-6.0 mm, 3.0-5.0 mm, or 3.0-4.0 mm. In some embodiments, the sample binding matrix has a thickness of about 4 mm.

The sample binding matrix may be placed at any position within the housing of the pipette tip. In some embodiments, the sample binding matrix is placed in close proximity of the inlet of the pipette tip so that the samples contact the binding matrix immediately after being taken into the housing of the pipette tip through its inlet. In one embodiment, the sample binding matrix is contiguous with the pipette tip inlet.

In some embodiments, the sample binding matrix is separated from the inlet by a distance of 0-40 mm, preferably 0-20 mm. In other embodiments, the sample binding matrix is separated from the inlet by a distance of 1-40 mm. In yet other embodiments, the sample binding matrix is separated from the inlet by a distance of 1-5 mm, 1-10 mm, 1-20 mm, 1-40 mm, 5-10 mm, 5-20 mm, or 5-40 mm.

In some embodiments, the sample binding matrix is fitted with an elastic collar to facilitate insertion into the housing. In these embodiments, the sample binding matrix can be any type of binding matrix, such as a sintered glass frit, a binding matrix made from other porous monolithic materials, a woven glass fiber binding matrix and a composite glass binding matrix. Insertion of a sample binding matrix into a pipette tip using a plastic or elastomeric collar or capsule around the perimeter of the binding matrix material has the advantage of enabling an easy and snug insertion without a requirement for heating the pipette tip or vacuum welding steps. The elastomer could be one or more of the following materials: natural rubber, nitrile, fluoroelastomer, silicone, ethylene propylene diene monomer, polyurethane, polybutadiene, neoprene or chloroprene, hydrogenated nitrile or highly saturated nitrile, perfluoroelastomer, AFLAS, fluorosilicone, butyl rubber, polyacrylic rubber, polyether block amides, ethylene-vinyl acetate, thermoplastic elastomer, thermoplastic olefin, styrene-ethylene-butylene-styrene, ethylene propylene rubber, epichlorohydrin. The sample binding matrix can be press fit into the elastomeric collar or the collar can be over-molded onto the sample binding matrix. In either case, the sample binding matrix may be a shape that is not cylindrical.

In some embodiments, the sample binding matrix (such as sintered glass frit, composite frit, or woven glass fiber binding matrix) is cut in cubic shape instead of standard disk geometry to reduce scrap rate. The cubic sample binding matrix is encased in a soft plastic or elastomeric cylindrical collar to form a binding matrix assembly, which is then press-fit into the housing. This assembly approach will result in more uniform insertion results (snug without scratches) without the need for heating or vacuum welding steps. This assembly process is also amenable to industry-established pipette tip assembly processes (i.e., frit cylinder insertion is made similar to aerosol binding matrix insertion).

The sample preparation device does not require centrifugation and thus eliminates the complexity associated with transferring samples from tubes to spin columns as well as simplifies the instrumentation required.

In addition, the vertical orientation of the sample preparation device during use forces bubbles to rise to the top of the device from the sample binding matrix, which improves fluidic control and enhances analyte binding and elution. Additionally, the small pores of the sample binding matrix reduces large air boluses into small bubbles which migrate to the top of the liquid column inside the sample passage way, creating a vibrant mixing effect of the binding buffer with the sample.

It should be noted that the pipette tip/syringe configurations of the sample preparation device allow bidirectional flow of the sample/washing/elution liquids through the sample binding matrix, while most sample preparation approaches rely on flow in only one direction through the binding matrix. The bidirectional flow feature not only allows the sample liquid to be taken into the sample preparation device and eluted out of the sample preparation device from the same opening (e.g., the second opening), but also permits a user to pipette a sample up and down for a number of cycles, thus providing the capability to process sample volumes larger than that of the sample preparation device.

### III. Method of Use

Another aspect of the present application relates to a method of purifying or isolating an analyte of interest with the sample preparation device of the present application.

In some embodiments, the method comprises the steps of passing a liquid samples through the sample binding matrix of the sample preparation device, wherein an analyte of interest in the sample binds specifically to the sample binding matrix, and releasing bound analyte of interest from the sample binding matrix with an elution solution. In some embodiments, the method further comprises the step of lysing cells or microorganisms in a biological sample to form the liquid sample. In some embodiments, the cells or microorganisms in the biological sample are lysed in a lysing solution with glass beads and a magnetic stirrer.

In some embodiments, the method comprises the steps of passing a liquid sample through the sample binding matrix in the tip of a syringe-like sample preparation device, wherein an analyte of interest in the sample binds specifically to the sample binding matrix, passing a washing solution through the sample binding matrix one or more times, and releasing bound analyte of interest from the sample binding matrix with an elution solution. In some embodiments, the method further comprises the step of lysing cells or microorganisms in a biological sample to form the liquid sample.

In some embodiments, the liquid sample and the washing solution are aspirated across the sample binding matrix and up into the overflow tube of the syringe-type sample preparation device of the present application. The overflow tube terminates inside the syringe body to allow waste to be stored in the syringe body cavity without flowing back into the binding matrix, and that allows the syringe plunger to be depressed (dispense mode) without allowing liquid flow back into the binding matrix (because of the overflow tube), when the syringe is held in an upright position. In some embodiments, the sample isolation process is automated to allow rapid process of samples.

### IV. Kits

Another aspect of the present application relates to a kit for sample preparation. In some embodiments, the kit comprises a sample binding tube, a pipette-tip shaped sample preparation device of the present application, one or more washing syringes pre-filled with a washing solution, an elution syringe pre-filled with an elution buffer and a sample collection tube. In some embodiments, the kit further comprises a drying syringe. The pre-filled syringes can be attached to and detached from the pipette-tip shaped sample preparation device during a sample preparation process.

In some embodiments, the kit comprises a syringe-type sample preparation device of the present application, and a reagent tray that contains a washing solution and elution solution. In some embodiments, the reagent tray further comprises a mixing chamber that may interface with a heater and/or cooler.

The following examples are offered by way of illustration of certain embodiments of aspects of the application herein. None of the examples should be considered limiting on the scope of the application.

### EXAMPLES

### Example 1

### DNA Sample Preparation With Tip-Type Sample Preparation Device and a Single Syringe

A device consisting of a porous glass binding matrix inserted into a pipette tip and attached to a 1 mL syringe via an adapter was evaluated for recovering genomic DNA from Escherichia coli broth culture. The device consisted of a 1.2 mL BioTix tip cut at the distal end and a 20 μL LTS Rainin tip. The aerosol filter is removed from the 20 μL tip and a 4.0 mm outer diameter glass SPT binding matrix is inserted into the proximal collar of the 20 μL tip. The 20 μL Rainin tip proximal collar is inserted onto the distal end of the 1.2 mL BioTix tip using a press fit. The E. coli broth culture was combined with a Lysis Buffer. The syringe aspirated this solution across the binding matrix and re-dispensed it back into the container five consecutive times. The syringe/pipette tip combination then toggled 1 mL of Akonni Wash Buffer J from a separate container back and forth across the binding matrix one time, followed by one aspiration-dispense cycle of the same procedure using 1 mL of Akonni Wash Buffer K in another container. Compressed air then purged the liquid remaining in the binding matrix, and lastly 25 μL of an elution buffer was dispensed on the binding matrix followed by a five minute wait step and 10 aspiration-dispense cycles across the binding matrix. The protocol was 16.75 minutes. The elution buffer was then analyzed on a Nanodrop and was found to be 2.1 for A260/A280 and 2.01 for A260/A230 with a concentration of 136 ng/μL.

### Example 2

### DNA Sample Preparation With Tip-Type Sample Preparation Device and Multiple Single Syringes

A device consisting of a porous composite silica binding matrix inserted into a pipette tip along with a series of syringes was evaluated for recovering genomic sinbus RNA virus from whole blood. Whole blood was spiked with 11,100 sindbis viral particles and incubated for 10 minutes with proteinase K. This sample was then combined with a Lysis Buffer and aspirated and dispensed with the pipette tip (including binding matrix) attached to a syringe. The pipette tip was then removed, and a syringe pre-loaded with Akonni Wash Buffer J was attached to the pipette tip, and the wash buffer was dispensed across the binding matrix. The same procedure was followed for Wash Buffer K and subsequently air (to dry the binding matrix). Lastly, an elution buffer was ejected from a syringe onto the binding matrix, incubated for 30 seconds and then dispensed through the frit. The recovered elution buffer was analyzed with a REV qPCR assay. Compared to the input, the elution buffer recovery was 83% and 82% for two replicate extractions.

Another embodiment for using the sample preparation tip of the present application is the following. The user packs single-use disposable kits that include all necessary reagents and consumables; no equipment is needed. Donning protective gear, the user takes a sample from the interrogated environment using a swab, a disposable transfer pipette, or a syringe. The sample is added to a collection container that allows pre-processing (e.g., lysis) and the output is introduced into a nucleic acid binding buffer. A syringe is used to flow sample and binding buffer through the sample binding matrix, which binds DNA from the sample. An additional syringe is used to wash the impurities from the matrix followed by another empty syringe, which dries the matrix. An alternative embodiment for drying is to use pressurized air such as in a carbon dioxide canister or pressurized air can or pre-pressurizing a syringe with the necessary fasteners to secure the plunger in place. The method of air drying may include a valve such as a one-way valve or a luer-activated valve. A final syringe is used to elute the nucleic acid from the matrix in preparation for further analysis (e.g., sequencing or library preparation).

### Example 3

### Field Portable Sample Preparation Kit With a Single Syringe

A 12-mL syringe was modified to include a cannula that traversed the outlet hole of the syringe. The cannula is secured in the luer fitting of the syringe and ends at the 2.5 mL mark of the 12-mL syringe. The cannula is a polyethylene tube that has an 0.13″ outer diameter and 0.063″ inner diameter. A duroshore 65A polyvinylchloride tube (0.156″ outer diameter and 0.085″ inner diameter) press fits over the distal end of the cannula. The other (distal) end of the PVC tube houses a 2.85 mm silica binding matrix. Another modification is the introduction of a pin that pierces the plunger and prevents the plunger from being fully depressed. The workflow is described in Table 1. The elution output is free of hemoglobin (observed by transparent color) and contains concentrated DNA eluted from the DNA binding matrix.

TABLE 1
Workflow for sample preparation in Example 3

| Step | Workflow Description | Typ. Stroke | | Speed | |
|---|---|---|---|---|---|
| 1 | Add blood to separate sample/lysis | n/a | | n/a | |
| | container, mix. Add IPA, mix (IPA | | | | |
| | may be pre-loaded in cap) | | | | |
| 2A | Aspirate pre-rinse | 3-4 | mL | 5 | mL/min |
| 2B | Aspirate sample + lysis buffer + IPA | 6-12 | mL | 1 | mL/min |
| Reset | Reset, extend plunger towards | reset 3 mL | | n/a | |
| | overflow | | | | |
| 2C | Aspirate Wash 1 | 3-5 | mL | 5 | mL/min |
| Reset | Reset, extend plunger towards | reset 3 mL | | n/a | |
| | overflow | | | | |
| 2D | Aspirate Wash 2 | 3-13 | mL | 5 | mL/min |
| 2E | Air Dry (N-cycles of aspirate/ | 3-13 | mL | 25 | mL/min |
| | dispense to dry binding matrix) | | | | |
| 2F | Aspirate elution buffer | 9-12 | mL | 1 | mL/min |
| 2G | Dispense elution buffer to Test Strip | 12-3 | mL | 5 | mL/min |

### Example 4

### Automated Syringe Sample Preparation

Another embodiment consists of integrating two design approaches: (1) a single dual-acting syringe 600 (FIG. 4) that interfaces with a serological-style (diaphragm) pump (pump 720 in FIG. 5) for course volume control and a precision piston (stepper motor 710 in FIG. 5) for fine volume control. The use of a diaphragm pump allows for high flow rates without an end limit (unlike conventional pipettes) which is beneficial for air drying; it has high linearity over a wide range of flow rates, which is advantageous for the range of volumes required for this application; and it does not require a plunger allowing for a small form factor. During the sample extraction stage the small piston 650 (shown in the insert of FIG. 4) does not enter the overflow tube 640, thus the diaphragm pump controls the flow rate across the binding matrix. When extraction is complete, a precision stepper motor advances the piston 650 into the overflow tube 640 and allows for fine volume control for sample analysis steps.

### Example 5

### Normalization

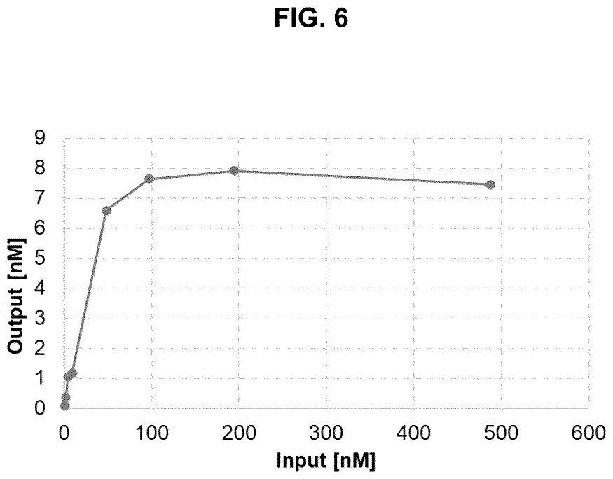

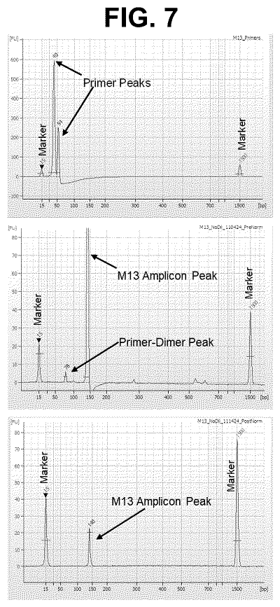

M13 bacteriophage DNA was PCR amplified, as an internal PCR control, using the KAPA Library Quantification Kit-Illumina/ABI Prism. A dilution series from stock (no dilution) to 400× of the amplified product were created to determine if the binding matrix saturates and thus could be used to normalize a range of input sequencing library concentrations. Each dilution of the amplified product (simulating a range of library DNA concentrations) was combined with a binding buffer. The mixture (input) was processed through the binding matrices, which were designed to have low binding capacity matrices so that it saturates below the expected concentrations of the PCR amplified libraries as found in FIG. 6. The undiluted (“dirtiest”) product from this study was analyzed on a Bioanalyzer to determine if binding matrix was also able to “cleanup” and isolate only the amplified product. As shown in FIG. 7, after normalization, there is only a single “clean” and narrow peak in the electropherogram at the expected molecular weight (140 bp), with no evidence of primer-dimer peaks, demonstrating successful PCR-cleanup. These data show that the binding matrix offers the potential to normalize sequencing libraries and do PCR cleanup with a single tip.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence, which is effective to meet the objectives there intended, unless the context specifically indicates the contrary.

1. A syringe-shaped sample preparation device, comprising:
a syringe body having a proximate end, a distal end and a syringe body cavity between the proximate end and the distal end;
a plunger located at the proximate end of the syringe body and movable along the syringe body cavity;
an overflow tube at the distal end of the syringe body and extending into the body cavity of the syringe body; and
sample purification tip at the distal end of the syringe body, wherein the sample purification tip comprises:
a housing that defines a sample passage-way between a first opening and a second opening, wherein the second opening is located outside of the body cavity of the syringe body; and
a sample binding matrix located within the sample passage-way of the housing, wherein the sample passage-way of the housing is in fluid communication with the syringe body cavity through the overflow tube.

2. The syringe-shaped sample preparation device of claim 1, wherein the sample purification tip is removable from the distal end of the syringe body.

3. The syringe-shaped sample preparation device of claim 1, wherein the sample binding matrix binds specifically to nucleic acids.

4. The syringe-shaped sample preparation device of claim 3, wherein the sample binding matrix comprises a composite frit.

5. The syringe-shaped sample preparation device of claim 4, wherein the composite frit comprises glass and a polymeric material.)

6. The syringe-shaped sample preparation device of claim 5, wherein the polymeric material is selected from the group consisting of polyethylene terephthalate, copolyesters, low-density polyethylene, high-density polyethylene, polyvinyl chloride, polypropylene, polystyrene, acrylonitrile-butadiene-styrene, acrylic methacrylate, polymethyl methacrylate, polycarbonate, polyurethane, nylon, polyethylene terephthalate glycol, polyetheretherketone, polytetrafluoroethylene, polyamide, polylactic acid, polyoxymethylene, polyether block amide, ethylene vinyl acetate, polyimide, polyphenylene sulfide, polysulfone, polyetherimide, fluorinated ethylene propylene, polyvinylidene fluoride, styrene-acrylonitrile, polybutylene terephthalate, polymethylpentene, polyether sulfone, polyphenylene oxide, epoxy, vinylester, polyester thermosetting plastic, phenol formaldehyde resins, acetal homopolymers and copolymers, polyethylene oxide, liquid crystal polymers, polyethylenimine, ultra high molecular weight polyethylene, cyclic olefin polymer, and cyclic olefin copolymers.

7. The syringe-shaped sample preparation device of claim 3, wherein the sample binding matrix is located in the proximity of the second opening of the sample purification tip.)

8. The syringe-shaped sample preparation device of claim 3, wherein the sample binding matrix is a composite frit with pore sizes in the range of 20-50 micron and a thickness in the range of 2-8 mm.

8. (canceled)

9. The syringe-shaped sample preparation device of claim 1, wherein the overflow tube extends 10-30 mm into the syringe body cavity from the distal end of the syringe body.

10. The syringe-shaped sample preparation device of claim 1, wherein the plunger comprises a stopper that prevents the plunger from fully entering the syringe body cavity.

11. A method for isolating an analyte from a biological sample, comprising the steps of:
lysing the biological sample in a lysis solution to form a liquid lysate;
passing the liquid lysate through the sample binding matrix of the sample analysis device of claim 1, wherein the analyte in the liquid lysate binds specifically to the sample binding matrix;
washing the sample binding matrix with a washing solution; and
releasing bound analyte from the sample binding matrix with an elution solution.

12. The method of claim 11, wherein the analyte is nucleic acid.)

13. A sample preparation device, comprising:
a housing defining a passage-way between a first opening and a second opening, wherein the housing is configured in the shape of a pipette tip; and
a sample binding matrix occupying a section of the passage-way, wherein the sample binding matrix comprises composite frit comprising glass and a polymeric material.

14. The sample preparation device of claim 13, wherein the sample binding matrix binds to nucleic acid.)

15. The sample preparation device of claim 13, wherein the sample binding matrix has a pore size in the range of 1 micron to 100 micron and a thickness in the range of 1 mm to 10.0 mm.

16. A method for isolating an analyte from a biological sample, comprising the steps of:
lysing the biological sample in a lysis solution to form a liquid lysate;
passing the liquid lysate through the sample binding matrix of the sample analysis device of claim 13, wherein the analyte in the liquid lysate binds specifically to the sample binding matrix;
washing the sample binding matrix with a washing solution; and
releasing bound analyte from the sample binding matrix with an elution solution.

17. A kit for isolating an analyte from a biological sample, comprising:
a syringe-shaped sample preparation device of claim 1;
a reagent pack; and
instructions for how to use the syringe-shaped sample preparation device with the reagent pack.

18. The kit of claim 17, wherein the reagent pack is a Blister-type reagent pack.

19. A kit for isolating an analyte from a sample, comprising:
a sample preparation device of claim 13;
one or more syringes pre-filled with reagents needed for isolating the analyte from the sample; and
instructions for how to use the sample preparation device with one or more syringes pre-filled with reagents.

20. A nucleic acid normalization device, comprising:
a housing defining a passage-way between a first opening and a second opening; and
a sample binding matrix occupies a section of the passage-way, wherein the sample binding matrix has a nucleic acid binding capacity that is saturated below an expected nucleic acid concentration in a sample.

21. The nucleic acid normalization device of claim 20, wherein the binding matrix has diameters in the range of 3-6 mm, a thickness in the range of 2-5 mm, and pore sizes in the range of 1-100 microns.

22. The nucleic acid normalization device of claim 21, wherein the first opening having a diameter that is greater than the diameter of the second opening, wherein the diameter of the binding matrix closer to the first opening has a diameter in the range of 3-6 mm, and wherein a side of the binding matrix is tapered toward the second opening of the binding matrix such that the diameter of the binding matrix closer to the second opening is less than the diameter closer to the first opening such that the binding matrix has a frustoconical shape to the contour of the passage-way.

23. A method to normalize nucleic acid in a sample, comprising the steps of:
loading the sample to the nucleic acid normalization device of claim 20, wherein the sample contains an expected nucleic acid concentration that exceeds the nucleic acid binding capacity of the binding matrix of the nucleic acid normalization device; and
eluting bound nucleic acid from the nucleic acid normalization device.
